(19) United States
(12) Patent Application Publication
(10) Pub. No.: US 2016/0207909 A1
(43) Pub. Date: Jul. 21, 2016

(54) Synthesis Method of Anti-Cancer Drug UK-1 and Derivatives Thereof

(71) Applicant: Yung-Tzung Huang, Kaohsiung (TW)

(72) Inventor: Yung-Tzung Huang, Kaohsiung (TW)

(21) Appl. No.: 14/964,651

(22) Filed: Dec. 10, 2015

(30) Foreign Application Priority Data
Jan. 20, 2015 (TW) 104101855

(51) Int. Cl. C07D 413/14; C07D 263/62

(52) U.S. Cl. CPC C07D 413/14; C07D 263/62

(57) ABSTRACT

A synthesis method of anti-cancer drug UK-1 includes: mixing methyl 2-amino-3-hydroxybenzoate, aldehyde and molecular sieves to form a mixture and firstly refluxing the mixture to form a first reactant; hydrolyzing the first reactant in a basic solution to form a hydrolyzed solution and secondly refluxing the hydrolyzed solution to form a second reactant; and dehydrating the second reactant and the methyl 2-amino-3-hydroxybenzoate in an acidic solution to form a dehydrated solution and thirdly refluxing the dehydrated solution to form an anti-cancer drug UK-1 or at least one derivative thereof.

### BACKGROUND OF THE INVENTION

1. Field of the Invention

The present invention relates to a synthesis method of anti-cancer drug UK-1 and derivatives thereof. Particularly, the present invention relates to the synthesis method of anti-cancer drug UK-1 and derivatives thereof suitable for lowering environmental pollution, reducing toxic preparation use and simplifying the process for mass production.

2. Description of the Related Art

By way of example, U.S. Pat. No. 7,294,643, entitled “UK-1 ANALOGUES: METHODS OF PREPARATION AND USE,” discloses a synthesis method of anti-cancer drug UK-1 and a series of analogues. However, the disclosed synthesis method includes five main synthetic steps for synthesizing the anti-cancer drug UK-1 and analogues, as best shown in FIG. 1, which is incorporated herein by reference.

Referring to the upper portion in FIG. 1, the synthesis method includes first synthetic step of: preparing compound 2.2 (2-(benzyloxy)benzoic acid) and compound 2.4 (2-(benzyloxy)benzoic acid), and utilizing CDI (1,1′-carbonyldiimidazole) dissolved in dry THF (tetrahydrofuran) for coupling reaction carboxylic acid functional group of compound 2.2 with amine functional group of compound 2.4 to form compound 2.3 (methyl 2-{[2-(benzyloxy)benzoyl]amino}-3-hydroxybenzoate).

With continued reference to the upper portion in FIG. 1, the synthesis method includes second synthetic step of: heating compound 2.3 at near 230° C. for a predetermined period of time for dehydration to form compound 2.5 (methyl 2-[2-(benzyloxy)phenyl]-1,3-benzoxazole-4-carboxylate).

Turning now to the right portion in FIG. 1, the synthesis method includes third synthetic step of: refluxing compound 2.5 in a solution, which contains THF (tetrahydrofuran), for base hydrolysis reaction (with 5M sodium hydroxide) to form compound 2.6 (2-[2-(benzyloxy)phenyl]-1,3-benzoxazole-4-carboxylic acid).

Turning now to the lower portion in FIG. 1, the synthesis method includes fourth synthetic step of: utilizing oxalyl chloride to transform carboxylic acid functional group of compound 2.6 into acyl chloride which is further reacted with compound 2.7 (methyl 2,3-diaminobenzoate) to form compound 2.8 (methyl 2-amino-3-({[2-(2-benzyloxyphenyl)-1,3-benzoxazol-4-yl]carbonyl}aminobenz-oate).

With continued reference to the lower portion in FIG. 1, the synthesis method includes fourth synthetic step of: refluxing compound 2.8 in a solution, which contains TsOH (p-toluenesulfonic acid) and toluene, to obtain compound 2.1 (Methyl 2-[2-(2-hydroxyphenyl)-1,3-benzoxazol-4-yl]-1H-benzimidazole-4-carboxylate, UK-1).

However, the above-mentioned synthesis method utilizes high toxic chemical substances and high-concentration strong basic solutions which will result in a high risk of environmental pollution in production. Additionally, the above-mentioned synthesis method further includes five main synthetic steps which further result in complicating the entire process and increasing manufacturing cost. Accordingly, the above-mentioned synthesis method is unsuitable for mass production of UK-1.

Hence, there is a need of improving the synthesis method of anti-cancer drug UK-1 for reducing the use of toxic chemical substances and strong bases and simplifying the entire process for mass production. The above-mentioned patent application publications are incorporated herein by reference for purposes including, but not limited to, indicating the background of the present invention and illustrating the situation of the art.

As is described in greater detail below, the present invention provides a synthesis method of anti-cancer drug UK-1 and derivatives thereof. In combination reaction, methyl 2-amino-3-hydroxybenzoate (compound 2), aldehyde (compound 3) and molecular sieves are mixed and refluxed to form a first reactant (compound 4) with an ester functional group. The ester functional group of compound 4 is hydrolyzed in a basic solution (or weak basic solution) and is further refluxed to form a second reactant (compound 5) with a carboxylic acid functional group. compound 5 and an equivalent weight of compound 2 are dehydrated in an acidic solution and are further refluxed to form UK-1 in such a way as to improve the conventional synthesis method of anti-cancer drug UK-1.

### SUMMARY OF THE INVENTION

The primary objective of this invention is to provide a synthesis method of anti-cancer drug UK-1 and derivatives thereof. In combination reaction, methyl 2-amino-3-hydroxybenzoate (compound 2), aldehyde (compound 3) and molecular sieves are mixed and refluxed to form a first reactant (compound 4) with an ester functional group. The ester functional group of compound 4 is hydrolyzed in a basic solution (or weak basic solution) and is further refluxed to form a second reactant (compound 5) with a carboxylic acid functional group. compound 5 and an equivalent weight of compound 2 are dehydrated in an acidic solution and are further refluxed to form UK-1. Advantageously, the synthesis method of the present invention is successful in reducing the use of toxic chemical substances and strong bases and simplifying the entire process.

The synthesis method of anti-cancer drug UK-1 and derivatives thereof in accordance with an aspect of the present invention includes:

Step A) mixing methyl 2-amino-3-hydroxybenzoate, aldehyde and molecular sieves to form a mixture and firstly refluxing the mixture to form a first reactant;

Step B) subsequently, hydrolyzing the first reactant in a basic solution to form a hydrolyzed solution and secondly refluxing the hydrolyzed solution to form a second reactant; and

Step C) finally, dehydrating the second reactant and the methyl 2-amino-3-hydroxybenzoate in an acidic solution to form a dehydrated solution and thirdly refluxing the dehydrated solution to form an anti-cancer drug UK-1 or at least one derivative thereof.

In a separate aspect of the present invention, the molecular sieves are 5A-type calcium molecular sieves or non-toxic molecular sieves.

In a further separate aspect of the present invention, the molecular sieves have a composition of: 0.80 CaO:0.20 Na₂O:1 Al₂O₃:2.0±0.1 SiO₂:x H₂O.

In yet a further separate aspect of the present invention, the methyl 2-amino-3-hydroxybenzoate, the aldehyde and the molecular sieves are mixed in an organic solvent.

In yet a further separate aspect of the present invention, the basic solution is a weak basic solution or a low-concentration basic solution.

In yet a further separate aspect of the present invention, the acidic solution is added to catalyze a reaction of the second reactant with an equivalent weight of the methyl 2-amino-3-hydroxybenzoate.

In yet a further separate aspect of the present invention, the mixture of methyl 2-amino-3-hydroxybenzoate, aldehyde and molecular sieves are firstly refluxed to form an ester functional group of the first reactant.

In yet a further separate aspect of the present invention, the ester functional group of the first reactant is hydrolyzed in the basic solution to form the hydrolyzed solution.

In yet a further separate aspect of the present invention, the hydrolyzed solution is secondly refluxed to form a carboxylic acid functional group of the second reactant.

Further scope of the applicability of the present invention will become apparent from the detailed description given hereinafter. However, it should be understood that the detailed description and specific examples, while indicating preferred embodiments of the invention, are given by way of illustration only, since various will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a schematic flow chart of a conventional synthesis method in accordance with the prior art.

FIG. 2 is a schematic flow chart of a synthesis method of anti-cancer drug UK-1 and derivatives thereof in accordance with a preferred embodiment of the present invention.

FIG. 3 is a chemical structure view of a first compound of anti-cancer drug UK-1 synthesized by the synthesis method in accordance with the preferred embodiment of the present invention.

FIG. 3A-3G are chemical structure views of derivatives of the first compound of anti-cancer drug UK-1 synthesized by the synthesis method in accordance with the preferred embodiment of the present invention.

FIG. 4 is a chemical structure view of a second compound of anti-cancer drug Me-UK-1 derivative synthesized by the synthesis method in accordance with the preferred embodiment of the present invention.

FIG. 4A-4K are chemical structure views of derivatives of the second compound of anti-cancer drug Me-UK-1 synthesized by the synthesis method in accordance with the preferred embodiment of the present invention.

FIG. 5 is a chemical structure view of a third compound of anti-cancer drug DeOH-UK-1 derivative synthesized by the synthesis method in accordance with the preferred embodiment of the present invention.

FIG. 5A-5Z are chemical structures of derivatives of the third compound of anti-cancer drug DeOH-UK-1 synthesized by the synthesis method in accordance with the preferred embodiment of the present invention.

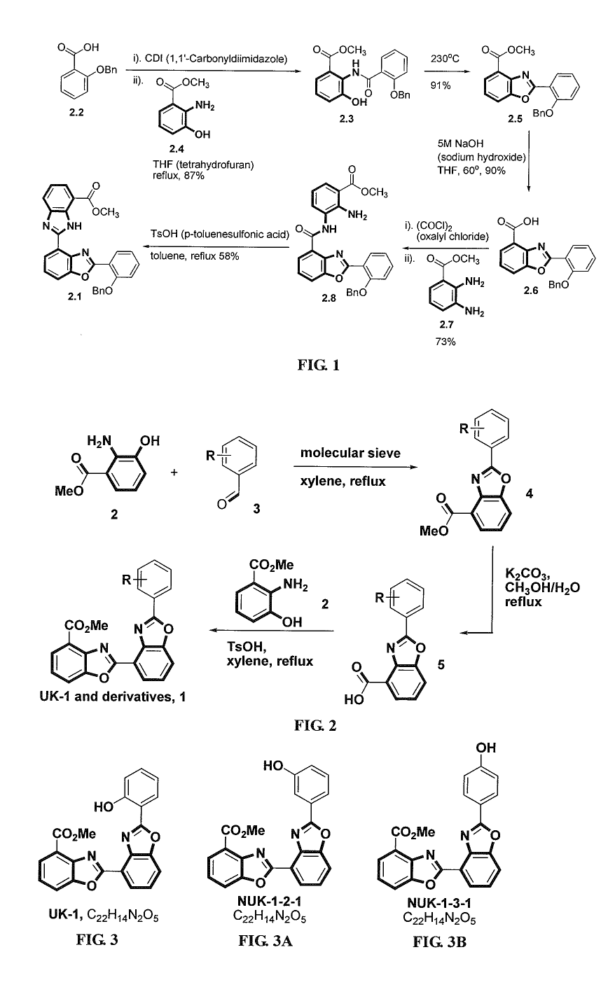

### DETAILED DESCRIPTION OF THE INVENTION

It is noted that a synthesis method in accordance with the preferred embodiment of the present invention can be applicable to prepare anti-cancer drug UK-1 and derivatives thereof. Generally, the anti-cancer drug UK-1 is a natural compound of mycelium extracted from streptomyces (sp. 517-02) for suppressing the growth of cancer cells.

The anti-cancer drug UK-1 derivatives thereof synthesized by the synthesis method in accordance with the preferred embodiment of the present invention has a chemical compound structure formed with heterocyclic compound, including two benzoxazoles of nitrogen and oxygen containing 5-membered-ring formation. However, the conventional synthesis method requires five main synthetic steps which will result in complicating the entire process, increasing the use of chemical reagents in activity test and manufacturing total cost.

The synthesis method of anti-cancer drug UK-1 and a series of derivatives thereof in accordance with the preferred embodiment of the present invention only require three synthetic steps which will reduce the use of chemical reagents in activity test and provide a manner to lower the risk of environmental pollution. Advantageously, the synthesis method of the present invention is applicable to various international green chemistry standards or requirements, such as “design of chemical products and processes that reduce or eliminate the use and generation of hazardous substances”. By way of example, the twelve principles of green chemistry includes prevention, atom Economy, less hazardous chemical syntheses, designing safer chemicals, safer solvents and auxiliaries, design for energy efficiency, use of renewable feedstocks, reduce derivatives, catalysis, design for degradation, real-time analysis for pollution prevention and inherently safer chemistry for accident prevention.

The phosphorescent stabilizer in accordance with the preferred embodiment of the present invention can be used as a stabilizer, an absorbent or a dispersion carrier for phosphorescent materials or are applicable to materials, displays, optoelectronics, biomedical engineering or other technical field, which are not limitative of the present invention.

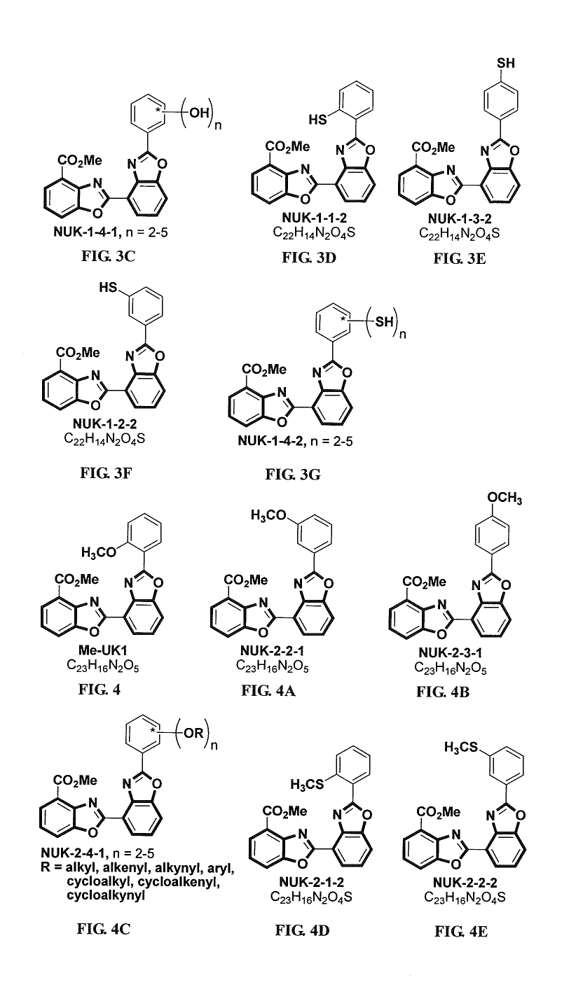

FIG. 2 shows a schematic flow chart of a synthesis method of anti-cancer drug UK-1 and derivatives thereof in accordance with a preferred embodiment of the present invention. Referring now to FIG. 2, the synthesis method of anti-cancer drug UK-1 includes Step A: mixing methyl 2-amino-3-hydroxybenzoate (compound 2), aldehyde (compound 3) and molecular sieves to form a mixture and firstly refluxing the mixture to form a first reactant (compound 4).

In a preferred embodiment, the molecular sieves are 5A-type calcium molecular sieves, non-toxic molecular sieves or other sieves. The molecular sieves have a composition of: 0.80 CaO:0.20 Na₂O:1 Al₂O₃:2.0±0.1 SiO₂:x H₂O. The non-toxic molecular sieves may not release toxic heavy metal and a mixture of molecular sieves with water has a degree of pH 8 or higher than 7 and less than 8. In another preferred embodiment, compound 2, compound 3 and molecular sieves are mixed in an organic solvent.

Still referring to FIG. 2, the synthesis method of anti-cancer drug UK-1 includes Step B: subsequently, hydrolyzing the first reactant formed with an ester functional group in a basic solution (strong or weak basic solutions) to form a hydrolyzed solution and secondly refluxing the hydrolyzed solution to form a second reactant (compound 5). In a preferred embodiment, the basic solution is selected from a low-concentration basic solution. In another preferred embodiment, the hydrolyzed solution is secondly refluxed to form a carboxylic acid functional group of compound 5.

Still referring to FIG. 2, the synthesis method of anti-cancer drug UK-1 includes Step C: finally, dehydrating compound 5 (second reactant) and an equivalent weight of compound 2 (methyl 2-amino-3-hydroxybenzoate) in an acidic solution to form a dehydrated solution and thirdly refluxing the dehydrated solution to form an anti-cancer drug UK-1 (compound 1) or at least one derivative thereof. In a preferred embodiment, the acidic solution is selected from a low-concentration acidic solution.

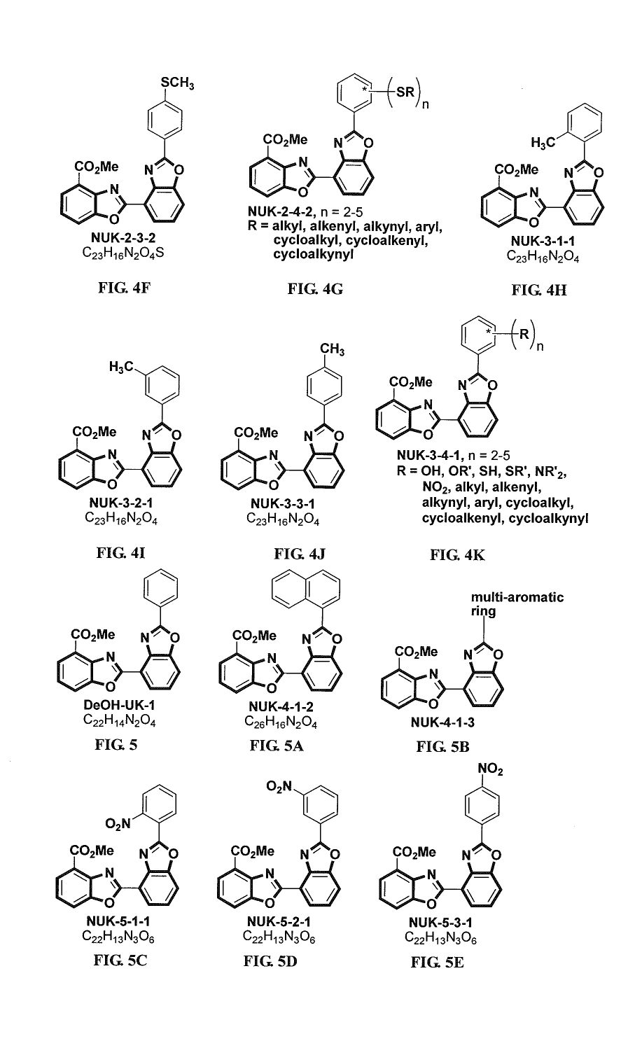

FIG. 3 shows a chemical structure view of a first compound of anti-cancer drug UK-1 synthesized by the synthesis method in accordance with the preferred embodiment of the present invention, as shown in FIG. 2. Referring to FIG. 3, the first compound of anti-cancer drug UK-1 possesses the molecular structure C₂₂H₁₄N₂O₅.

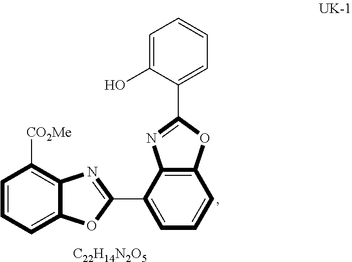

FIG. 3A-3F show chemical structure views of a series of derivatives of the first compound of anti-cancer drug UK-1 synthesized by the synthesis method in accordance with the preferred embodiment of the present invention, as shown in FIG. 2. In a preferred embodiment, a derivative of the first compound of anti-cancer drug UK-1 possesses the structural formula NUK-1-2-1.

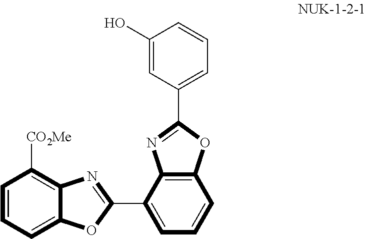

In another preferred embodiment, a derivative of the first compound of anti-cancer drug UK-1 possesses the structural formula NUK-1-3-1.

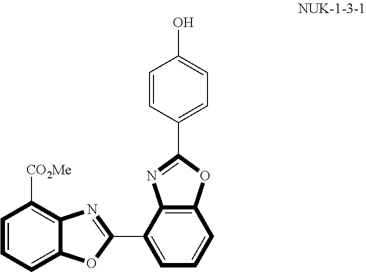

In another preferred embodiment, a derivative of the first compound of anti-cancer drug UK-1 possesses the structural formula NUK-1-4-1.

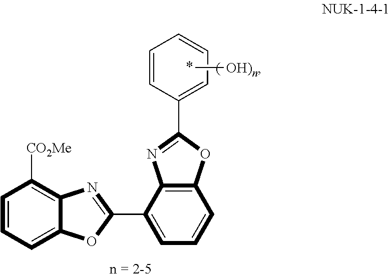

where n is 2 to 5.

In another preferred embodiment, a derivative of the first compound of anti-cancer drug UK-1 possesses the structural formula NUK-1-1-2.

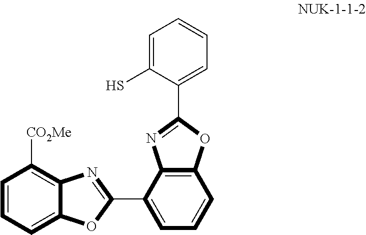

In another preferred embodiment, a derivative of the first compound of anti-cancer drug UK-1 possesses the structural formula NUK-1-3-2.

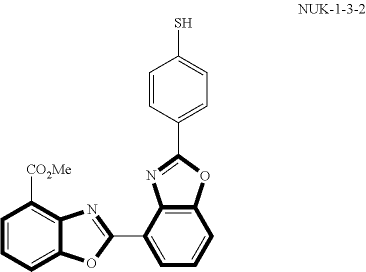

In another preferred embodiment, a derivative of the first compound of anti-cancer drug UK-1 possesses the structural formula NUK-1-2-2.

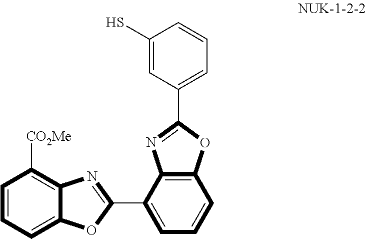

In another preferred embodiment, a derivative of the first compound of anti-cancer drug UK-1 possesses the structural formula NUK-1-4-2.

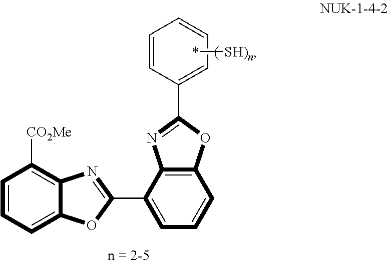

where n is 2 to 5.

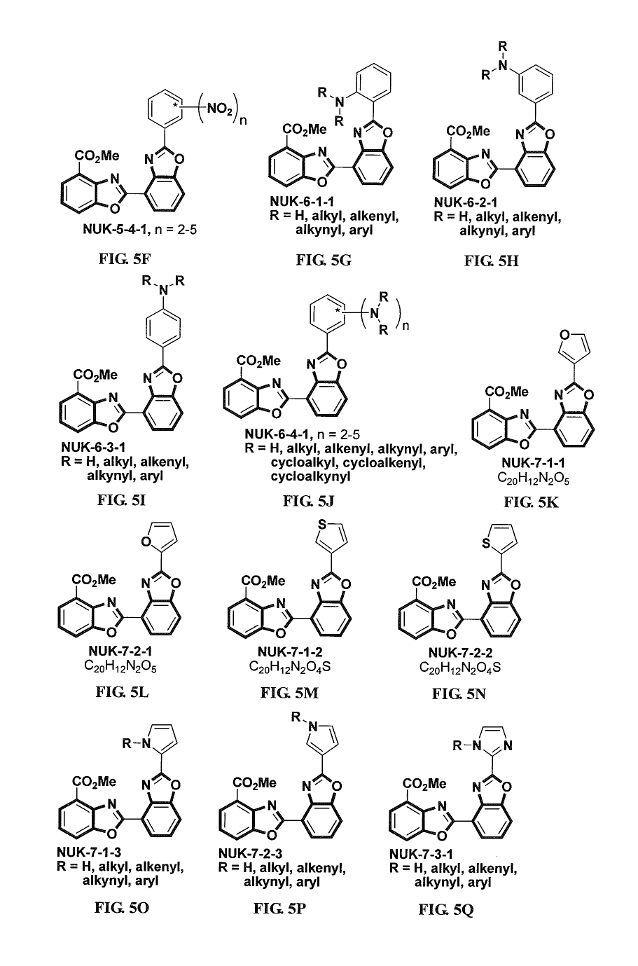

FIG. 4 shows a chemical structure view of a second compound of anti-cancer drug Me-UK-1 derivative synthesized by the synthesis method in accordance with the preferred embodiment of the present invention, as shown in FIG. 2. Referring to FIG. 4, the second compound of anti-cancer drug Me-UK-1 possesses the molecular structure C₂₃H₁₆N₂O₅.

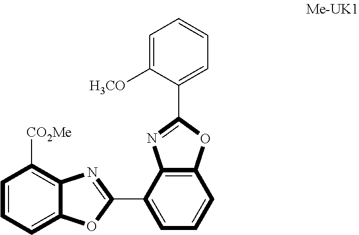

FIG. 4A-4K show chemical structure views of a series of derivatives of the second compound of anti-cancer drug UK-1 synthesized by the synthesis method in accordance with the preferred embodiment of the present invention, as shown in FIG. 2. In a preferred embodiment, a derivative of the second compound of anti-cancer drug Me-UK-1 possesses the structural formula NUK-2-2-1.

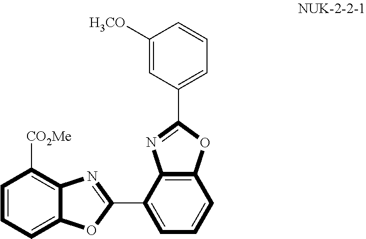

In another preferred embodiment, a derivative of the second compound of anti-cancer drug Me-UK-1 possesses the structural formula NUK-2-3-1.

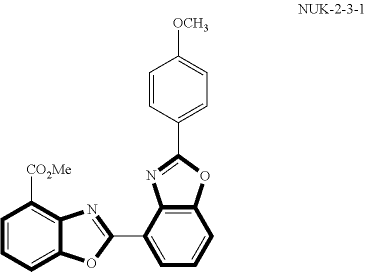

In another preferred embodiment, a derivative of the second compound of anti-cancer drug Me-UK-1 possesses the structural formula NUK-2-4-1.

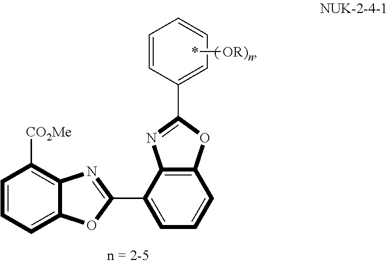

where R is alkyl, alkenyl, alkynyl, aryl, cycloalkyl, cycloalkenyl or cycloalkynyl; and where n is 1 to 5.

In another preferred embodiment, a derivative of the second compound of anti-cancer drug Me-UK-1 possesses the structural formula NUK-2-1-2.

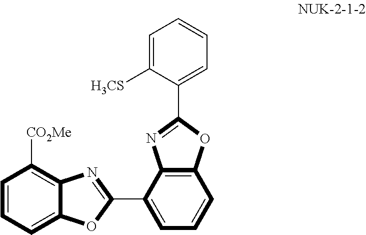

In another preferred embodiment, a derivative of the second compound of anti-cancer drug Me-UK-1 possesses the structural formula NUK-2-2-2.

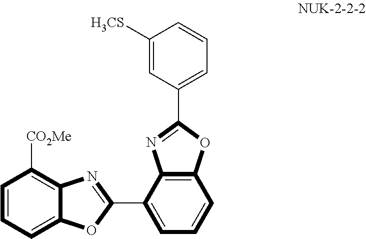

In another preferred embodiment, a derivative of the second compound of anti-cancer drug Me-UK-1 possesses the structural formula NUK-2-3-2.

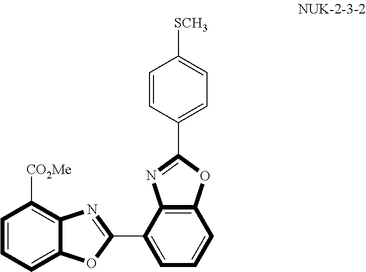

In another preferred embodiment, a derivative of the second compound of anti-cancer drug Me-UK-1 possesses the structural formula NUK-2-4-2.

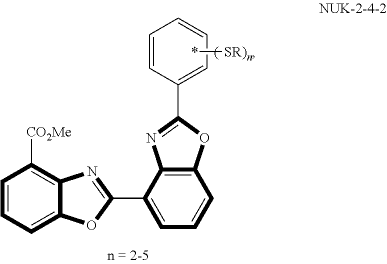

where R is alkyl, alkenyl, alkynyl, aryl, cycloalkyl, cycloalkenyl or cycloalkynyl; and where n is 1 to 5.

In another preferred embodiment, a derivative of the second compound of anti-cancer drug Me-UK-1 possesses the structural formula NUK-3-1-1.

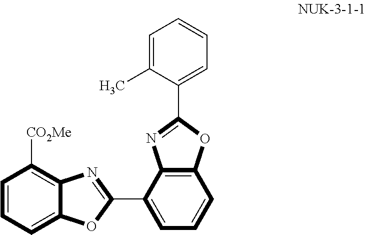

In another preferred embodiment, a derivative of the second compound of anti-cancer drug Me-UK-1 possesses the structural formula NUK-3-2-1.

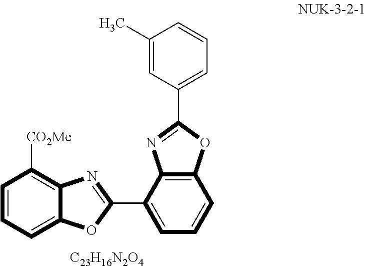

In another preferred embodiment, a derivative of the second compound of anti-cancer drug Me-UK-1 possesses the structural formula NUK-3-3-1.

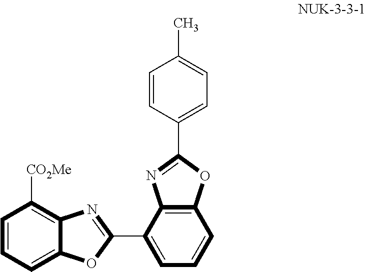

In another preferred embodiment, a derivative of the second compound of anti-cancer drug Me-UK-1 possesses the structural formula NUK-3-4-1.

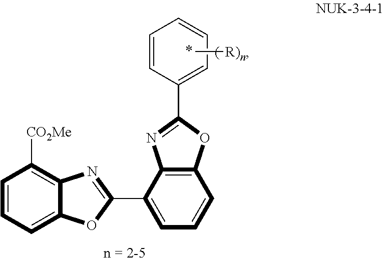

where R is OH, OR′, SH, SR′, NR′₂, NO₂, alkyl, alkenyl, alkynyl, aryl, cycloalkyl, cycloalkenyl or cycloalkynyl; and where n is 1 to 5.

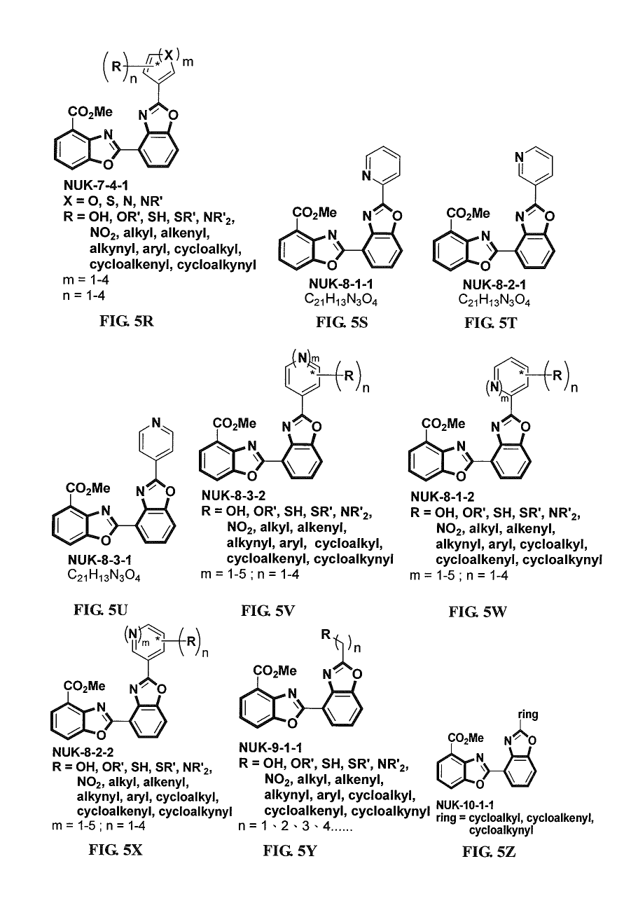

FIG. 5 shows a chemical structure view of a third compound of anti-cancer drug DeOH-UK-1 derivative synthesized by the synthesis method in accordance with the preferred embodiment of the present invention, as shown in FIG. 2. Referring to FIG. 5, the third compound of anti-cancer drug DeOH-UK-1 possesses the molecular structure C₂₂H₁₄N₂O₄.

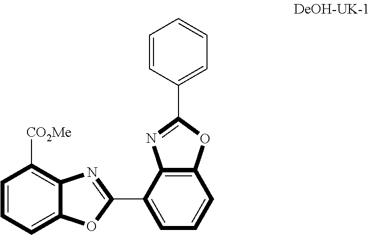

FIG. 5A-5Z show chemical structures of a series of derivatives of the third compound of anti-cancer drug DeOH-UK-1 synthesized by the synthesis method in accordance with the preferred embodiment of the present invention. In a preferred embodiment, a derivative of the third compound of anti-cancer drug DeOH-UK-1 possesses the structural formula NUK-4-1-2.

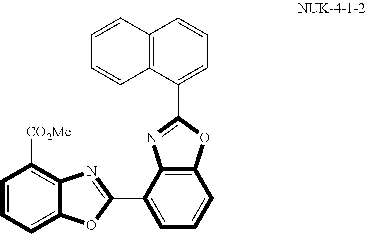

In another preferred embodiment, a derivative of the third compound of anti-cancer drug DeOH-UK-1 possesses the structural formula NUK-4-1-3.

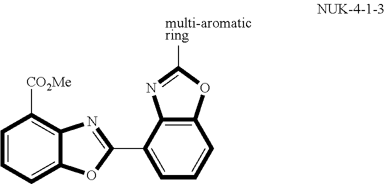

where ring is multi-aromatic ring.

In another preferred embodiment, a derivative of the third compound of anti-cancer drug DeOH-UK-1 possesses the structural formula NUK-5-1-1.

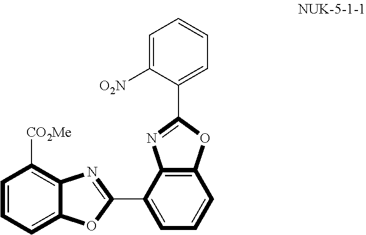

In another preferred embodiment, a derivative of the third compound of anti-cancer drug DeOH-UK-1 possesses the structural formula NUK-5-2-1.

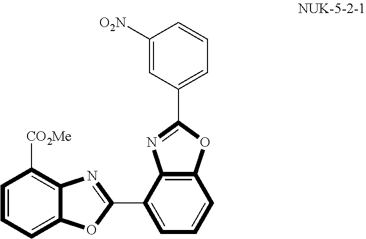

In another preferred embodiment, a derivative of the third compound of anti-cancer drug DeOH-UK-1 possesses the structural formula NUK-5-3-1.

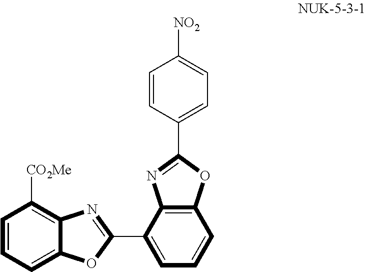

In another preferred embodiment, a derivative of the third compound of anti-cancer drug DeOH-UK-1 possesses the structural formula NUK-5-4-1.

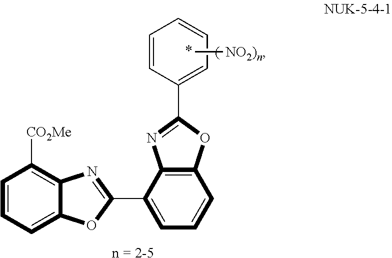

where n is 1 to 5.

In another preferred embodiment, a derivative of the third compound of anti-cancer drug DeOH-UK-1 possesses the structural formula NUK-6-1-1.

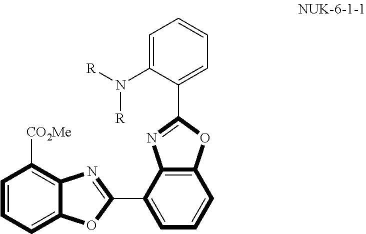

where R is H, alkyl, alkenyl, alkynyl or aryl.

In another preferred embodiment, a derivative of the third compound of anti-cancer drug DeOH-UK-1 possesses the structural formula NUK-6-2-1.

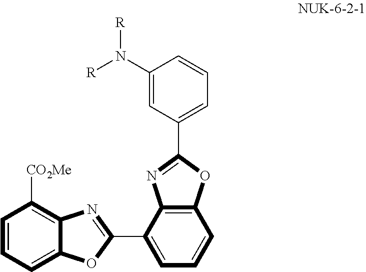

where R is H, alkyl, alkenyl, alkynyl or aryl.

In another preferred embodiment, a derivative of the third compound of anti-cancer drug DeOH-UK-1 possesses the structural formula NUK-6-3-1.

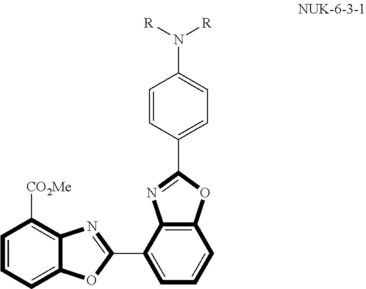

where R is H, alkyl, alkenyl, alkynyl or aryl.

In another preferred embodiment, a derivative of the third compound of anti-cancer drug DeOH-UK-1 possesses the structural formula NUK-6-4-1.

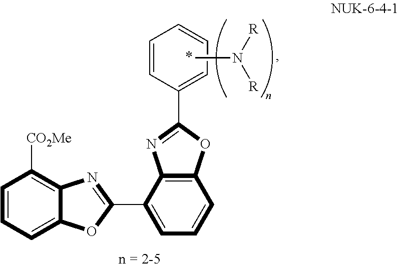

where R is H, alkyl, alkenyl, alkynyl, aryl, cycloalkyl, cycloalkenyl or cycloalkynyl; and where n is 1 to 5.

In another preferred embodiment, a derivative of the third compound of anti-cancer drug DeOH-UK-1 possesses the structural formula NUK-7-1-1.

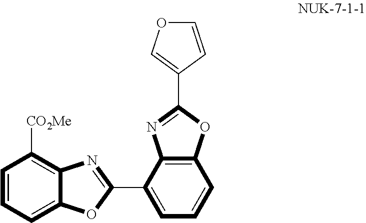

In another preferred embodiment, a derivative of the third compound of anti-cancer drug DeOH-UK-1 possesses the structural formula NUK-7-2-1.

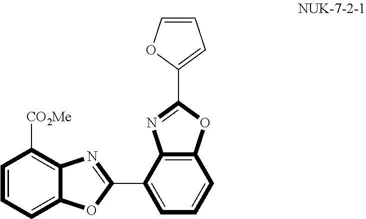

In another preferred embodiment, a derivative of the third compound of anti-cancer drug DeOH-UK-1 possesses the structural formula NUK-7-1-2.

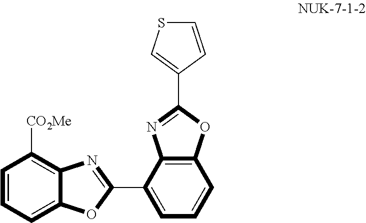

In another preferred embodiment, a derivative of the third compound of anti-cancer drug DeOH-UK-1 possesses the structural formula NUK-7-2-2.

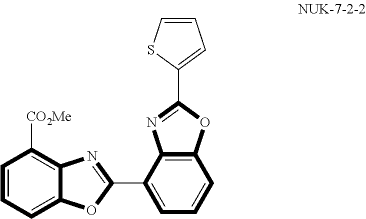

In another preferred embodiment, a derivative of the third compound of anti-cancer drug DeOH-UK-1 possesses the structural formula NUK-7-1-3.

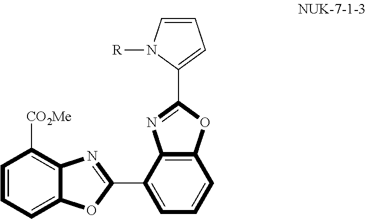

where R is H, alkyl, alkenyl, alkynyl or aryl.

In another preferred embodiment, a derivative of the third compound of anti-cancer drug DeOH-UK-1 possesses the structural formula NUK-7-2-3.

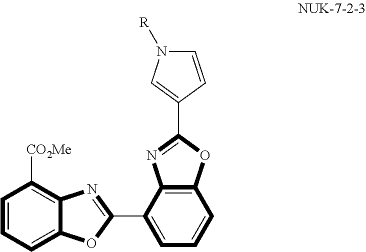

where R is H, alkyl, alkenyl, alkynyl or aryl.

In another preferred embodiment, a derivative of the third compound of anti-cancer drug DeOH-UK-1 possesses the structural formula NUK-7-3-1.

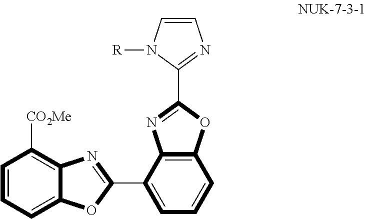

where R is H, alkyl, alkenyl, alkynyl or aryl.

In another preferred embodiment, a derivative of the third compound of anti-cancer drug DeOH-UK-1 possesses the structural formula NUK-7-4-1.

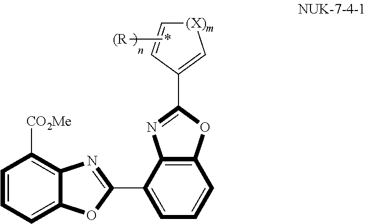

where O is O, S or NR′₂; where R is OH, OR′, SH, SR′, NR′₂, NO₂, alkyl, alkenyl, alkynyl, aryl, cycloalkyl, cycloalkenyl or cycloalkynyl; and where m is 1 to 4 and n is 1 to 4.

In another preferred embodiment, a derivative of the third compound of anti-cancer drug DeOH-UK-1 possesses the structural formula NUK-8-1-1.

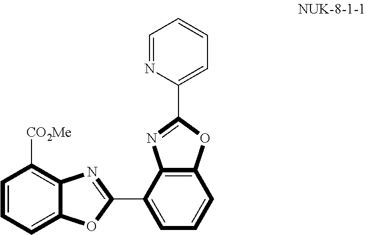

In another preferred embodiment, a derivative of the third compound of anti-cancer drug DeOH-UK-1 possesses the structural formula NUK-8-2-1.

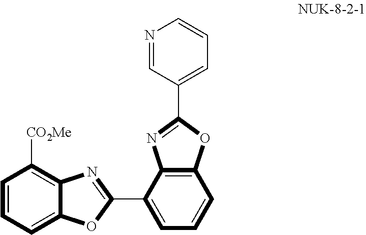

In another preferred embodiment, a derivative of the third compound of anti-cancer drug DeOH-UK-1 possesses the structural formula NUK-8-3-1.

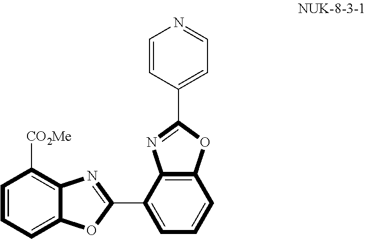

In another preferred embodiment, a derivative of the third compound of anti-cancer drug DeOH-UK-1 possesses the structural formula NUK-8-3-2.

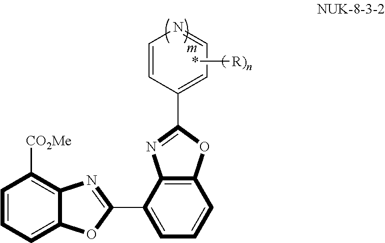

where R is OH, OR′, SH, SR′, NR′₂, NO₂, alkyl, alkenyl, alkynyl, aryl, cycloalkyl, cycloalkenyl or cycloalkynyl; and where m is 1 to 4 and n is 1 to 4.

In another preferred embodiment, a derivative of the third compound of anti-cancer drug DeOH-UK-1 possesses the structural formula NUK-8-1-2.

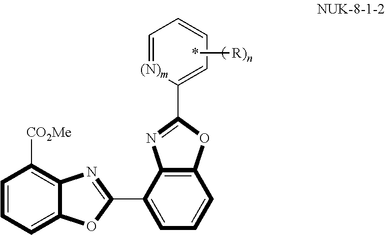

where R is OH, OR′, SH, SR′, NR′₂, NO₂, alkyl, alkenyl, alkynyl, aryl, cycloalkyl, cycloalkenyl or cycloalkynyl; and where m is 1 to 4 and n is 1 to 4.

In another preferred embodiment, a derivative of the third compound of anti-cancer drug DeOH-UK-1 possesses the structural formula NUK-8-2-2.

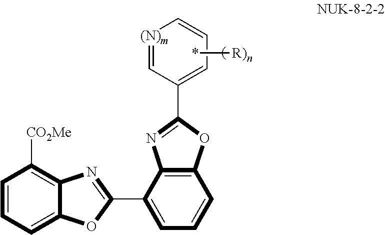

where R is OH, OR′, SH, SR′, NR′₂, NO₂, alkyl, alkenyl, alkynyl, aryl, cycloalkyl, cycloalkenyl or cycloalkynyl; and where m is 1 to 4 and n is 1 to 4.

In another preferred embodiment, a derivative of the third compound of anti-cancer drug DeOH-UK-1 possesses the structural formula NUK-9-1-1.

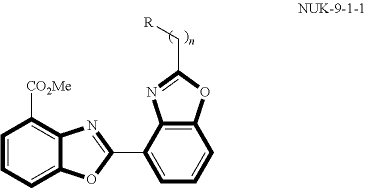

where R is OH, OR′, SH, SR′, NR′₂, NO₂, alkyl, alkenyl, alkynyl, aryl, cycloalkyl, cycloalkenyl or cycloalkynyl; and where n is 1, 2, 3, 4 to m.

In another preferred embodiment, a derivative of the third compound of anti-cancer drug DeOH-UK-1 possesses the structural formula NUK-10-1-1.

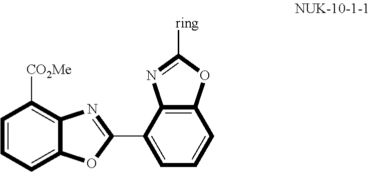

where ring is cycloalkyl, cycloalkenyl or cycloalkynyl.

Although the invention has been described in detail with reference to its presently preferred embodiment, it will be understood by one of ordinary skills in the art that various modifications can be made without departing from the spirit and the scope of the invention, as set forth in the appended claims.

What is claimed is:

1. A synthesis method comprising:
mixing methyl 2-amino-3-hydroxybenzoate, aldehyde and molecular sieves to form a mixture and firstly refluxing the mixture to form a first reactant;
subsequently, hydrolyzing the first reactant in a basic solution to form a hydrolyzed solution and secondly refluxing the hydrolyzed solution to form a second reactant; and
finally, dehydrating the second reactant and the methyl 2-amino-3-hydroxybenzoate in an acidic solution to form a dehydrated solution and thirdly refluxing the dehydrated solution to form an anti-cancer drug UK-1 or at least one derivative thereof.

2. The synthesis method as defined in claim 1, wherein the molecular sieves are 5A-type calcium molecular sieves or non-toxic molecular sieves.

3. The synthesis method as defined in claim 1, wherein the molecular sieves have a composition of: 0.80 CaO:0.20 Na₂O:1 Al₂O₃:2.0±0.1 SiO₂:x H₂O.

4. The synthesis method as defined in claim 1, wherein the methyl 2-amino-3-hydroxybenzoate, the aldehyde and the molecular sieves are mixed in an organic solvent.

5. The synthesis method as defined in claim 1, wherein the basic solution is a weak basic solution or a low-concentration basic solution.

6. The synthesis method as defined in claim 1, wherein the acidic solution is added to catalyze a reaction of the second reactant with an equivalent weight of the methyl 2-amino-3-hydroxybenzoate.

7. The synthesis method as defined in claim 1, wherein the mixture of methyl 2-amino-3-hydroxybenzoate, aldehyde and molecular sieves are firstly refluxed to form an ester functional group of the first reactant.

8. The synthesis method as defined in claim 7, wherein the ester functional group of the first reactant is hydrolyzed in the basic solution to form the hydrolyzed solution.

9. The synthesis method as defined in claim 1, wherein the hydrolyzed solution is secondly refluxed to form a carboxylic acid functional group of the second reactant.

10. The synthesis method as defined in claim 1, wherein the derivative has the structural formula Me-UK-1 or DeOH-UK-1

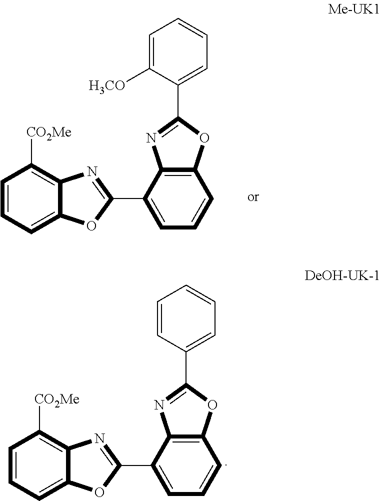

11. A chemical compound of anti-cancer drugs comprising:
a derivative of a compound having the structural formula UK-1, Me-UK-1 or DeOH-UK-1

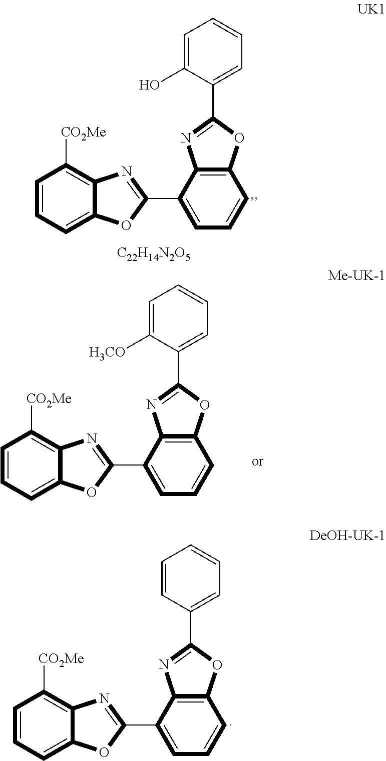

12. The chemical compound as defined in claim 11, wherein the derivative of the compound UK-1 has the structural formula, including 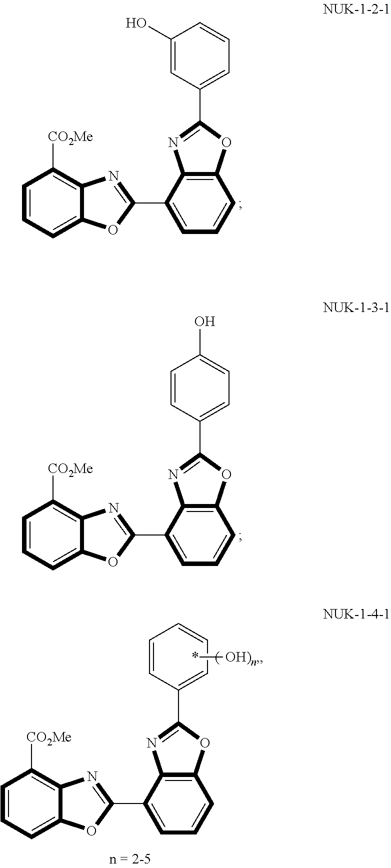
where n is 2 to 5; 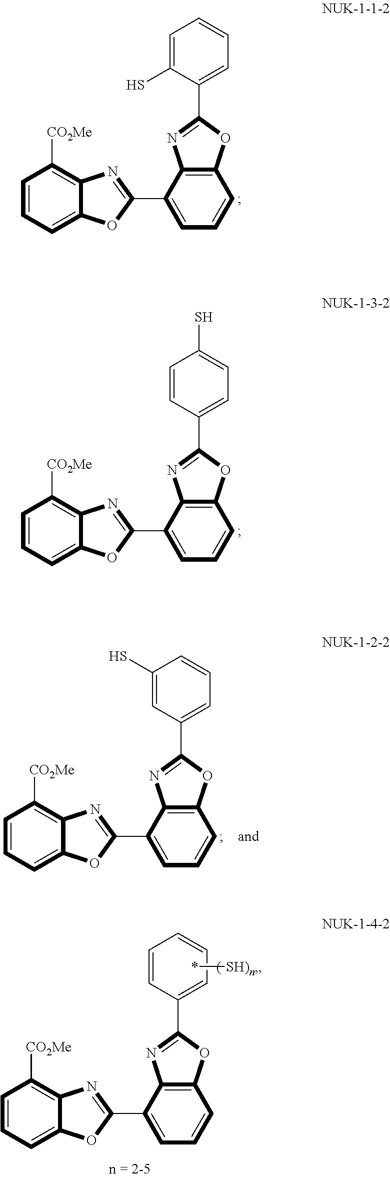
where n is 2 to 5.

13. The chemical compound as defined in claim 11, wherein the derivative of the compound Me-UK-1 has the structural formula, including 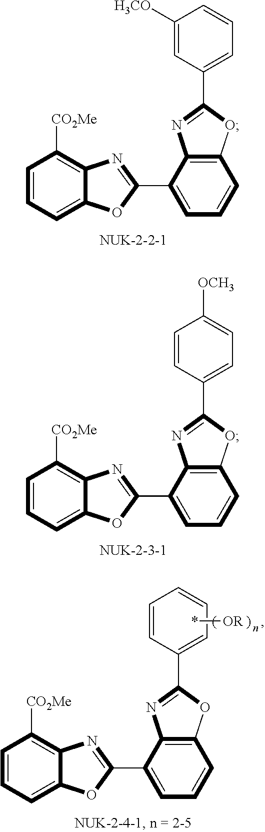
where R is alkyl, alkenyl, alkynyl, aryl, cycloalkyl, cycloalkenyl or cycloalkynyl; and where n is 1 to 5; 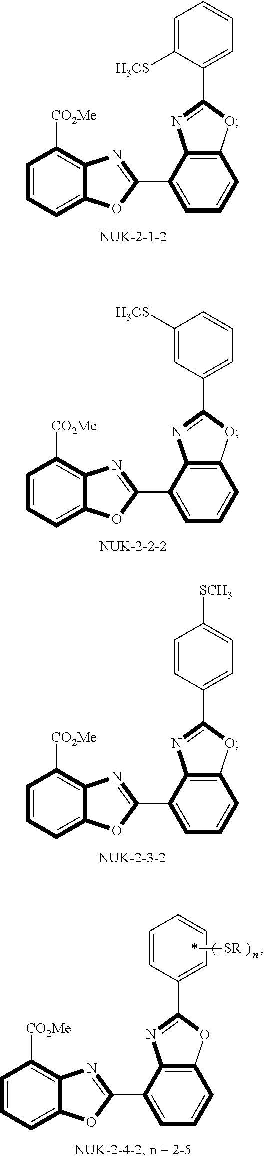
where R is alkyl, alkenyl, alkynyl, aryl, cycloalkyl, cycloalkenyl or cycloalkynyl; and where n is 1 to 5; 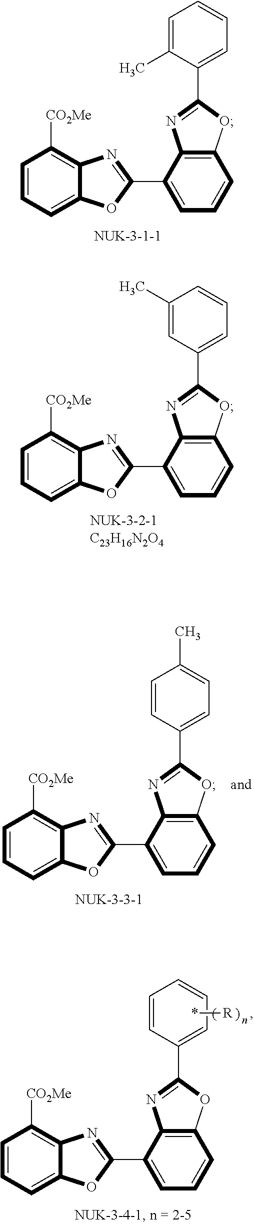
where R is OH, OR′, SH, SR′, NR′₂, NO₂, alkyl, alkenyl, alkynyl, aryl, cycloalkyl, cycloalkenyl or cycloalkynyl; and where n is 1 to 5.

14. The chemical compound as defined in claim 11, wherein the derivative of the compound DeOH-UK-1 has the structural formula, including 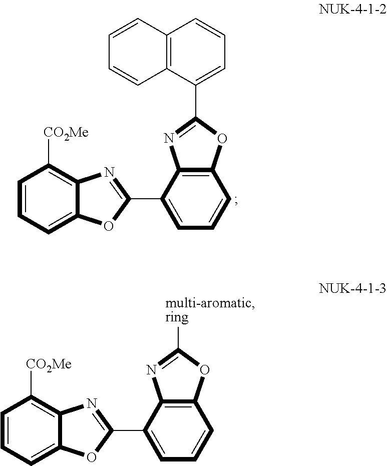
where ring is multi-aromatic ring; 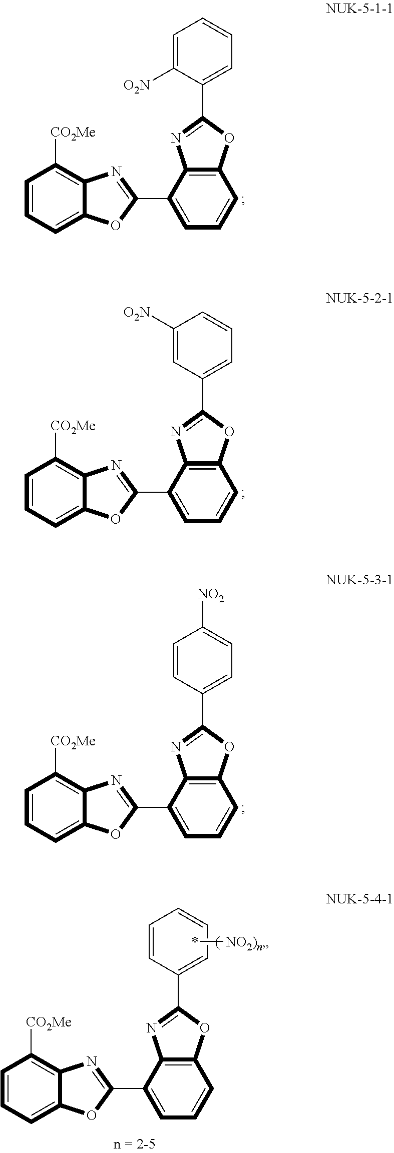
where n is 1 to 5; 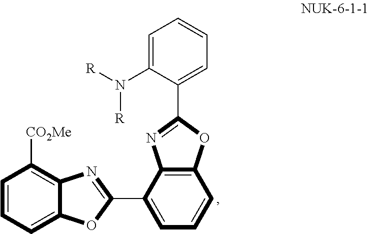
where R is H, alkyl, alkenyl, alkynyl or aryl; 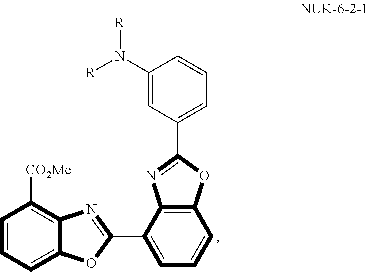
where R is H, alkyl, alkenyl, alkynyl or aryl; 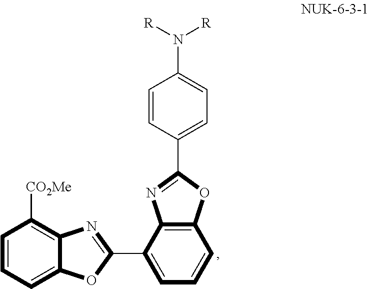
where R is H, alkyl, alkenyl, alkynyl or aryl; 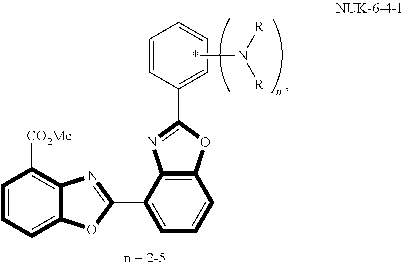
where R is H, alkyl, alkenyl, alkynyl, aryl, cycloalkyl, cycloalkenyl or cycloalkynyl; and where n is 1 to 5; 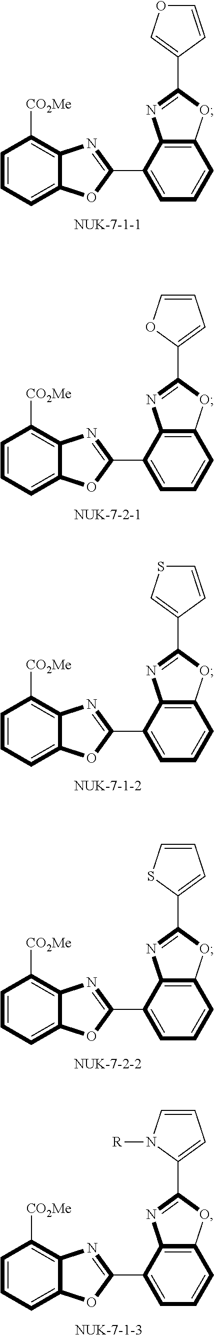
where R is H, alkyl, alkenyl, alkynyl or aryl; 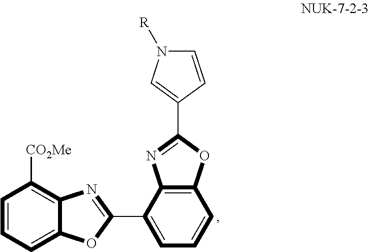
where R is H, alkyl, alkenyl, alkynyl or aryl; 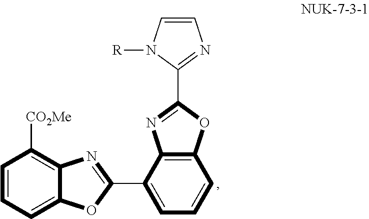
where R is H, alkyl, alkenyl, alkynyl or aryl; 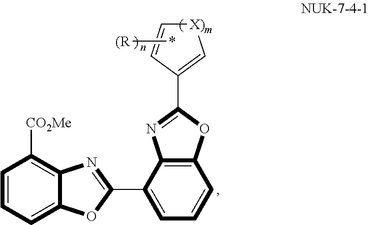
where O is O, S or NR′₂; where R is OH, OR′, SH, SR′, NR′₂, NO₂, alkyl, alkenyl, alkynyl, aryl, cycloalkyl, cycloalkenyl or cycloalkynyl; and where m is 1 to 4 and n is 1 to 4; 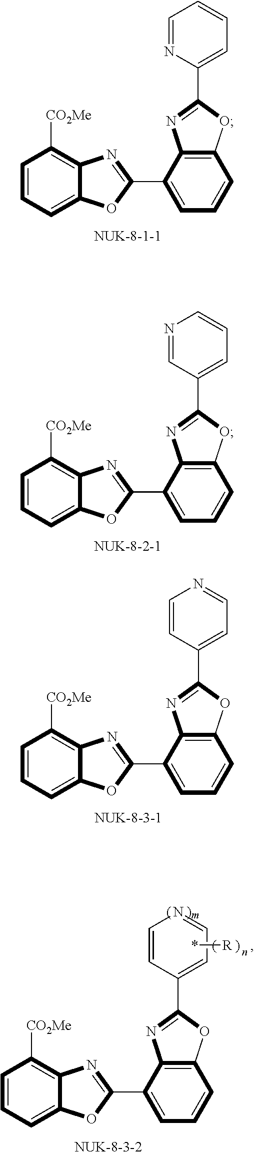
where R is OH, OR′, SH, SR′, NR′₂, NO₂, alkyl, alkenyl, alkynyl, aryl, cycloalkyl, cycloalkenyl or cycloalkynyl; and where m is 1 to 4 and n is 1 to 4; 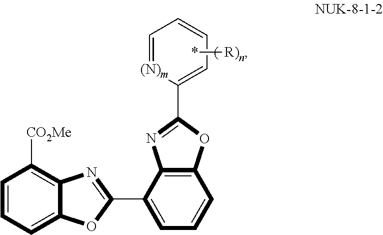
where R is OH, OR′, SH, SR′, NR′₂, NO₂, alkyl, alkenyl, alkynyl, aryl, cycloalkyl, cycloalkenyl or cycloalkynyl; and where m is 1 to 4 and n is 1 to 4; 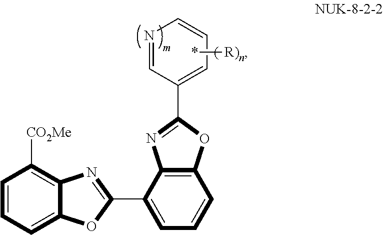
where R is OH, OR′, SH, SR′, NR′₂, NO₂, alkyl, alkenyl, alkynyl, aryl, cycloalkyl, cycloalkenyl or cycloalkynyl; and where m is 1 to 4 and n is 1 to 4; 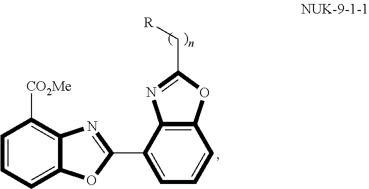
where R is OH, OR′, SH, SR′, NR′₂, NO₂, alkyl, alkenyl, alkynyl, aryl, cycloalkyl, cycloalkenyl or cycloalkynyl; and where n is 1, 2, 3, 4 to m; and 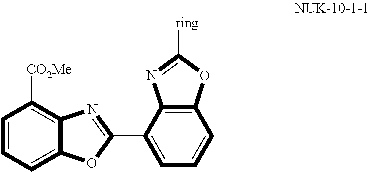
where ring is cycloalkyl, cycloalkenyl or cycloalkynyl.
